(19) United States
(12) Patent Application Publication
(10) Pub. No.: US 2025/0195009 A1
(43) Pub. Date: Jun. 19, 2025

(54) METHOD FOR RECOVERING A NOISE CONTAMINATED SIGNAL OBTAINED FROM A MONITORING DEVICE

(71) Applicant: Buddi Limited, Rickmansworth (GB)

(72) Inventors: Ewan FINDLAY, Milnathort (GB); David Ming Hui FOO, Milnathort (GB)

(21) Appl. No.: 18/986,618

(22) Filed: Dec. 18, 2024

(30) Foreign Application Priority Data
Dec. 19, 2023 (GB) 2319492.1

(51) Int. Cl. A61B 5/00; A61B 5/1477; H04W 64/00

(52) U.S. Cl. CPC A61B 5/7203; A61B 5/0022; A61B 5/1477; A61B 5/4845; A61B 5/7246; A61B 5/725; A61B 5/7253; H04W 64/006

(57) ABSTRACT

A method of processing a detection signal contaminated with noise is presented. The detection signal is a signal for detecting a substance ingested by a subject and has a signal profile formed of a peak and a decaying slope. The method includes the steps of: reading the detection signal, detecting noise in the detection signal; identifying if the detection signal is recoverable based on one or more characteristics of the noise in the signal profile; and applying a signal recovery algorithm to recover the detection signal.

### CROSS REFERENCE TO RELATED APPLICATIONS

The present application claims priority from GB Patent Application No. 2319492.1, filed Dec. 19, 2023, which is hereby incorporated by reference in its entirety as if fully set forth below and for all applicable purposes.

### TECHNICAL FIELD

The present disclosure relates to a method for recovering a noise contaminated signal obtained from a monitoring device for detecting a substance ingested by a subject. The present disclosure also relates to a monitoring system and/or device configured process and recover such a noise contaminated detection signal.

### BACKGROUND

Transdermal alcohol detection is an established means of monitoring the alcohol intake status of a subject. Such detection devices require close proximity to the subject's skin in order to read a detection signal profile, for instance through an ankle or wrist strap, the air-alcohol-concentration is measured directly from the air in the direction of the subject's skin. These transdermal alcohol detection devices can also be configured to transmit data to a remote server system. Transdermal alcohol detection devices can further be configured to have dual functionality of measuring the subject's intake of intoxicating substances as well as reporting the location of the device. The location can be derived from global positioning system (GPS) data, global system for mobile communications (GSM) data or a radio frequency (RF) beacon. Both the location data and the detection signal may be transmitted to a remote server via the GSM network or another wireless communications means.

Having the dual functionality of location and alcohol intake status of a subject provides several advantages. For example, if the subject is an offender on a judicial order and is known to have previously abused substances like alcohol, having this dual functionality offers a single solution for tracking and monitoring the offender. However, combining the functionality of reading a substance detection signal and wireless communications presents a problem. The high intensity signals used to communicate the data to a remote server interfere with the low intensity detection signal being collected from the sensor. Therefore the signal profile of the detection signal as read by the transdermal alcohol detection device is often contaminated with noise from the wireless communications system being used.

It is an object of the disclosure to address one or more of the above mentioned limitations.

### SUMMARY

According to a first aspect of the disclosure, there is provided a method of processing a detection signal contaminated with noise, wherein the detection signal is a signal for detecting a substance ingested by a subject; the method comprising: reading the detection signal, wherein the signal has a signal profile formed of a peak and a decaying slope; detecting noise in the detection signal; identifying if the detection signal is recoverable based on one or more characteristics of the noise in the signal profile; and applying a signal recovery algorithm to recover the detection signal.

Optionally, the signal recovery algorithm comprises applying a low pass filter to obtain a low frequency component and a high frequency component.

Optionally, the signal recovery algorithm comprises splitting the detection signal into several windows; comparing the signal profile of the detection signal with a template signal in each window; calculating in each window a correlation number; and restoring the signal in any window in which the correlation number is less than a threshold value.

Optionally, a series of correlation numbers are calculated using a piecewise correlation function.

Optionally, the detection signal is restored in a window by replacing the detection signal in that window with the template signal in that window.

Optionally, the signal recovery algorithm comprises: performing a temporal transform of the detection signal from a time domain to a frequency domain to obtain a frequency converted signal comprising a set of frequency bands ordered from a lowest frequency to a highest frequency; and removing a set of preidentified frequency bands from the frequency converted signal.

For instance, the set of preidentified frequency bands may be high frequency bands above a threshold frequency.

Optionally, the temporal transform is a Fast Fourier Transform or a Wavelet Transform.

Optionally, the signal recovery algorithm comprises fitting a custom function to the detection signal to obtain a level of noise contamination in the detection signal.

Optionally, the method comprises assigning a label to the noise or to the detected signal, wherein the label indicates at least one of a location of the noise along the signal profile, information indicating whether the detection signal is recoverable or not recoverable, and a level of accuracy of a recovered signal.

Optionally, noise characteristics comprise noise location and noise intensity.

Optionally, the detection signal is recoverable if the noise is located outside of a predefined window. For instance, the predefined window may start about a rising edge of the detection signal and stop at a given percentage of the decay. For example, the predefined window may stop at about 10% of the decay, or between 10% and 20% of the decay.

Optionally, the detection signal is an electrochemical signal.

Optionally, the substance is alcohol.

Optionally, the noise comprises interference arising from communication devices.

Optionally, the method comprises estimating an uncertainty of the recovered signal.

According to a second aspect of the disclosure, there is provided a monitoring system for use on a subject, the monitoring system comprising: a monitoring device for use on a subject comprising a sensor configured to sense a detection signal for detecting a substance ingested by the subject, wherein the detection signal has a signal profile formed of a peak and a decaying slope; and a communication interface; the system further comprising a processor configured to process the detection signal according to the method of the first aspect of the disclosure.

Optionally, the processor is provided as part of the monitoring device.

Optionally, the communication interface is configured to transmit the recovered detection signal to a remote server.

Optionally, the processor is provided on a remote server, the communication interface being configured to transmit raw data to the remote server for processing.

Optionally, the method comprises a geolocation tracker configured to provide a location signal of the monitoring device.

Optionally, the communications interface comprises a wireless communications interface.

### DESCRIPTION OF THE DRAWINGS

The disclosure is described in further detail below by way of example and with reference to the accompanying drawings, in which:

FIG. 1 is a method of processing a detection signal according to the present disclosure;

FIG. 2 is a block diagram of an example embodiment of a device that implements the method of FIG. 1;

FIG. 3 is a plot of a detection signal free of noise;

FIG. 4A is a plot of an example detection signal where the signal profile is contaminated with noise;

FIG. 4B is a plot of another example detection signal where the signal profile is contaminated with noise;

FIG. 4C is a plot of a third example detection signal where the signal profile is contaminated with noise;

FIG. 5 is a flow chart of an example signal recovery algorithm that can be implemented in the method of FIG. 1;

FIG. 6A is a profile view of an example embodiment of wearable device for implementing the method of FIG. 1;

FIG. 6B is another profile view of the wearable device of FIG. 6A.

### DESCRIPTION

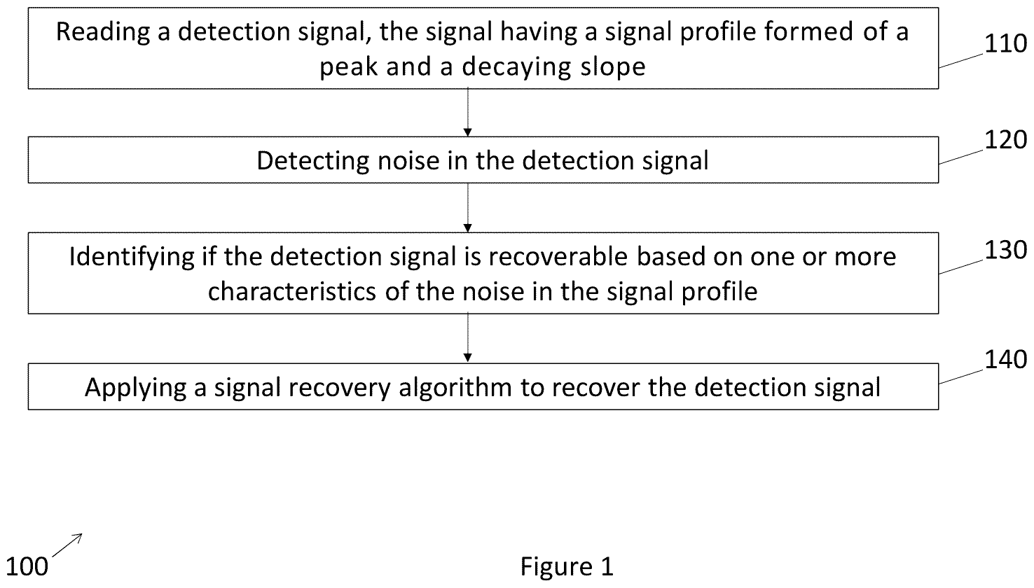

FIG. 1 illustrates a method of processing a detection signal contaminated with noise. The detection signal is a signal for detecting a substance, such as alcohol, ingested by a subject.

In step 110, a reading of the detection signal is performed. The detection signal has a signal profile which is formed of a peak and a decaying slope. For example, the detection signal may be an electrochemical signal.

Noise is detected in the detection signal at step 120. For instance, the noise may comprise interferences arising from communication devices such as Global System for Mobile Communications (GSM) or WiFi. Step 110 takes a finite time to be performed, for example 60 seconds, during which time the GSM modem may transmit a number of signals to communicate with a bases station, either to convey information or to verify its GSM cell-status. The interferences can show in several ways in the signal profile, such as through dips, spikes or a combination of the two.

At step 130, an identification of whether the detection signal is recoverable is performed based on one or more characteristics of the noise in the signal profile. For instance noise characteristics may include noise location and noise intensity among others. At this step, a label may be assigned to the noise. Such a label may be used to indicate the location of the noise in the signal. The label may also be used to indicate the accuracy of any applied noise removal applied to it in step 140.

At step 140, a signal recovery algorithm is applied to recover the detection signal.

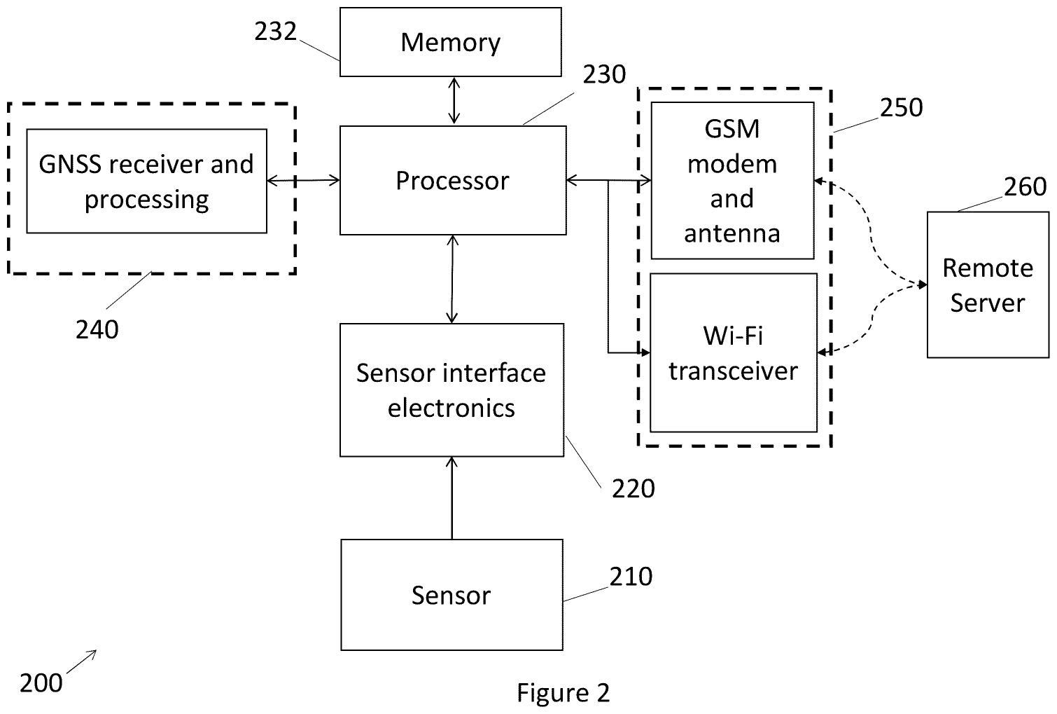

FIG. 2 is a block diagram of an example device 200 that implements the method of FIG. 1. The device comprises a sensor 210, a processor 230, a memory 232, a communication interface 250 and a remote server 260. The sensor 210 may be coupled to the processor via an interface circuit 220 for performing readout of the sensor 210. The interface circuit 220 may include an amplifier and an ADC circuit to transform the very weak signal from the sensor into a digital one for processing. A buffer may be provided between the ADC and the bus to the processor. The sensor 210 may be an electrochemical sensor.

The sensor 210 is configured to produce a detection signal driven by the amount of substance presented to it and is indicative of the amount of substance ingested by a subject. The substance could be, for example, alcohol and the detection signal could be an electrochemical signal obtained by an electrochemical sensor. The detection signal has a signal profile formed of a peak and a decaying slope. The detection signal as detected by the sensor 210 is passed to the processor 230 via the sensor interface electronics 220. The interface circuit 220 is configured to transform the weak signal from the sensor into a digital signal for processing and/or packaging by the processor 230. The processor 230 may be configured to carry out the steps of the method of FIG. 1. In this case, the processor 230 would read the detection signal and detects noise in the detection signal; identify if the detection signal is recoverable based on the location of the noise in the signal profile and then applies a signal recovery algorithm to recover the detection signal. Alternatively the raw data may be packaged and transmitted to a remote server for processing.

The device 200 may also include a geolocation tracker 240, configured to provide a location signal of the subject. The geolocation tracker 240 may include a global navigation satellite system (GNSS) receiver and processor, for instance, a Global Positioning System (GPS) or other types of satellite-based positioning, navigation and timing systems. The geolocation tracker 240 provides the location signal to the processor 230.

It will be appreciated that the processor 230 may be configured to execute various algorithms to perform noise rejection. For instance this may include executing a machine learning algorithm for performing noise rejection.

The recovered detection signal and the location signal are transmitted to the communications interface 250. The communication interface 250 may include a GSM modem and antenna or a Wi-Fi transceiver or a combination of both. In alternative embodiments the communications interface 250 could comprise one or more of the following: Bluetooth, Wi-FI, GSM, and near-field wireless communication devices. The communication interface 250 is configured to transmit the recovered detection signal and the location data wirelessly to the remote server 260. In alternative embodiments, the raw data can be packaged and transmitted to the remote server 260, and the noise detection and signal recovery algorithm performed on the server 260.

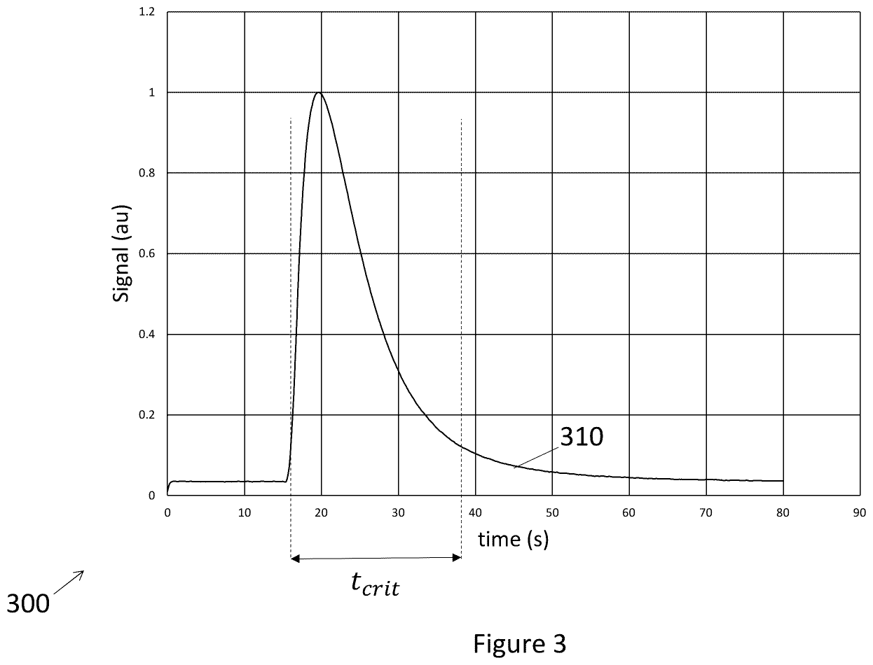

FIG. 3 is a plot showing a detection signal for detecting a substance ingested by a subject. The signal profile 310 in this plot is not contaminated with noise. The signal profile has a baseline value, followed by a clearly defined peak at 20s. After which time, the signal profile decays back down towards the baseline. This is the typical shape of a detection signal for an electrochemical sensor.

The reading derived from a signal profile 310 that is not contaminated by noise is given by the following summation:

$$Reading = \frac{1}{{f}_{s}} {\sum }_{n = 1}^{N} ( {y}_{n} - {y}_{0} ) \qquad ( 1 )$$

Where the summation starts at a start trigger to start the reading and stops at an end trigger giving the Nth value, with fₛ being the sampling frequency.

The reading interval is time Δt and the value of the signal at a time tₙ is yₙ.

The trigger can be a timed, amplitude or proportionally derived trigger. For example, the start trigger can be timed from the reading initiation point, in other words the zero time point on the graph. Alternatively, the start trigger can be set when the signal amplitude rises above a certain level. The end trigger can be, for example, timed from either the reading initiation point or from the amplitude peak. Alternatively, the end trigger can be set to be the point where the signal drops below a preset proportion of the signal peak.

The baseline signal for subtraction is represented by value y₀. The derivation of the baseline signal, y₀, can be done in a number of ways. For example, the average value may be taken over a portion of the signal between the reading initiation and the start of the substance sampling to derive the baseline value y₀. The substance sampling can be seen around t=16 s on plot 300. Alternatively, given that some electrochemical sensors can shift their baseline on processing a chemical input, the baseline value can be generated post measurement by taking the average value of a portion of the signal tail. For example, the average value may be obtained for the last 10 seconds preceding the reading end (t=80 s on the graph). The peak and decaying slope of the detection signal profile occur within a critical time window t_{crit}.

The critical time window t_{crit} may be set based on the type of sensor and/or the age of the sensor and should be long enough for performing a reading under standard conditions. For instance, the critical time window may start about the rising edge of the detection signal and stop at a given percentage of the decay, for example, it may stop at about 10% of the decay, or between 10% and 20% of the decay.

The critical time window t_{crit} contains the information between the signal peak and the tail that permits the prediction of the rest of the signal, by extrapolation or interpolation. However, large contamination of the peak or both areas that can be used to generate a baseline will basically reduce the veracity of the integral to nil.

For the signal profile 310, the sensor takes about Δt≈80 s to read the detection signal and the critical time window occurs at t_{crit}≈15 s-40 s. During the reading interval Δt, the communications interface, for example a GSM modem, may transmit a signal several times for different purposes. This will result in interference in the signal, or noise contamination. For GSM interference, the noise can take several forms. For example, a series of spikes which distort the reading over a period of up to 10 s. It may also appear as a dip in the signal reading due to the GSM modem's high consumption of battery power. The noise can also be a combination of both spikes and dips. The presence of noise in the signal profile 310 can be detected using a variety of standard filtering techniques. For example, comparison of the outputs of high frequency filtering and low frequency filtering, template comparison to spot anomalies in the signal, frequency domain signal analysis and time domain smoothing of the signal.

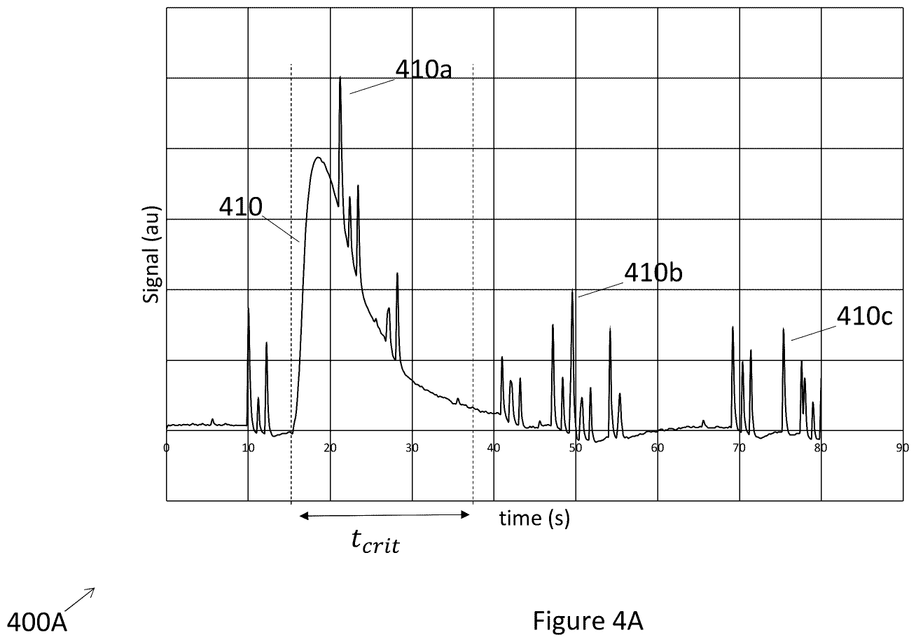

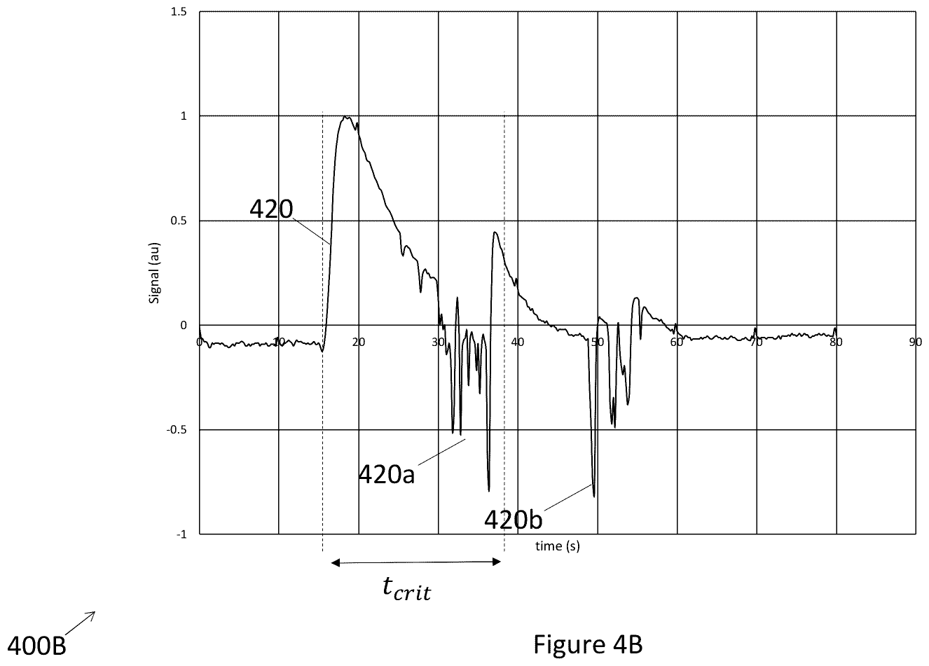

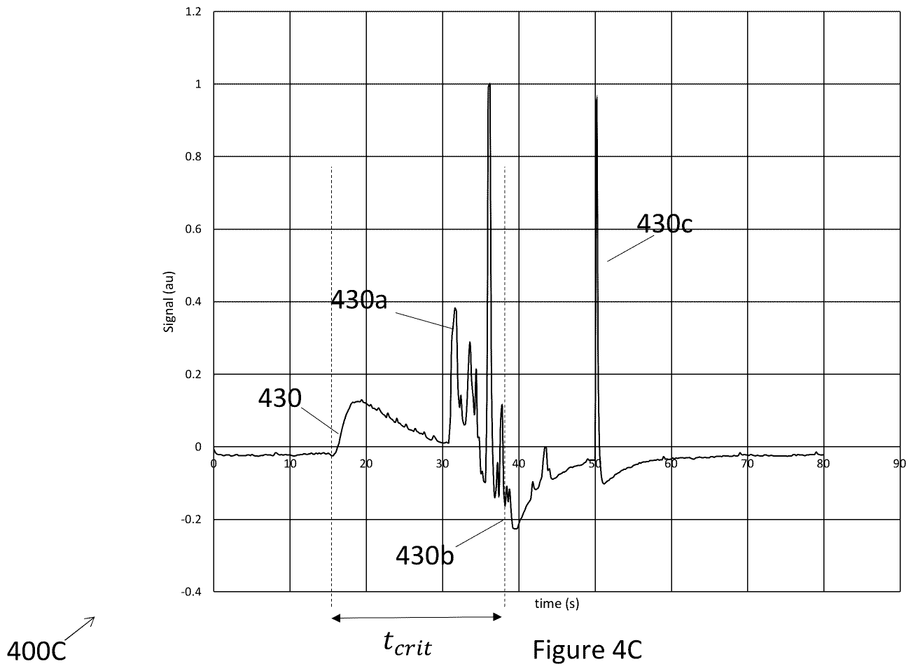

FIG. 4A shows an example signal profile 410 which has been contaminated with noise. The noise in signal 410 can be seen as groups of spikes in the signal profile as highlighted at 410a, 410b and 410c. These groups are located at different points along the signal profile 410. A valid baseline that can be determined for t<10 seconds. Low pass filtering can be used to remove most of the spike present within the critical time window t_{crit}, and the tail can be extrapolated. Consequently, this signal can be labeled as noisy, but recoverable as a reading answer can be given along with the warning.

FIG. 4B shows another example signal profile 420 which has been contaminated with noise. The noise in signal 420 can be seen as groups of dips in the signal profile as highlighted at 420a and 420b. These groups are located at different points along the signal profile 420. This signal can also be recovered. A stable baseline although slightly contaminated with spikes can be determined at the start and/or the end of the signal. The peak and initial exponential decay are available, and smoothing the section up to 30 s permits extrapolating. Such a signal may be labelled as noisy but recoverable.

FIG. 4C shows a third example signal profile 430 which has been contaminated with noise. The noise in signal 430 can be seen as a combination of dips and spikes as highlighted at 430a, 430b and 430c which are located at different points along the signal profile 420.

The location of where the noise contaminates the signal profile indicates the times at which the interference occurred during the reading interval Δt. This location determines whether the detection signal can be recovered by the signal recovery algorithm. As GSM interference typically only has a duration of less than or approximately 10 s, there can be contaminated profiles where parts of the underlying signal profile are still available to allow for reconstruction of the detection signal. For example, a signal with noise that contaminates the tail end of the profile will be more easily recoverable than a signal where the noise contaminates the critical window t_{crit}. When the detection signal is checked for noise contamination, a label can be assigned to the noise to flag that the signal has been recovered and to indicate the position of the noise contamination, as well as the obvious unrecoverable case. Examples of the noise labels that can be assigned are:
00—No noise detected in the signal profile
11—Noise detected in the tail of the signal profile
12—Noise detected in the decaying slope of the signal profile
13—Noise detected near the peak of the signal profile
99—Signal profile not recoverable

For the noise label 99 indicating an unrecoverable signal, this could be because the noise obscures the signal beyond repair within the critical window t_{crit} or because the noise is masking multiple features of the profile. The noise labels given above are examples of labels that would be generated for a signal recovery algorithm that uses prior knowledge of the non-contaminated signal profile.

The signal profile 410 of FIG. 4A is primarily contaminated with noise in the tail end of the profile. Therefore, the detection signal would be identified as recoverable, as discussed above. However, signal profile 430 will be identified as unrecoverable as the noise is contaminating the critical window along the signal profile.

Overall, the possibility of recovering a signal with a good degree of accuracy depends on both the ability to define a signal baseline and the location of the noise within the critical window t_{crit}. Noise in both of the two baseline generation intervals has the potential to offset the reading from its real value, hence preventing recovery or reducing accuracy of the recovered signal. The presence of noise within the critical window t_{crit} can distort an attempt to recover the signal before integrating it. Therefore, the extent and position of the noise in the critical window t_{crit} also drives the potential for recovery. The signal recovery algorithm used will also influence whether a signal is recoverable.

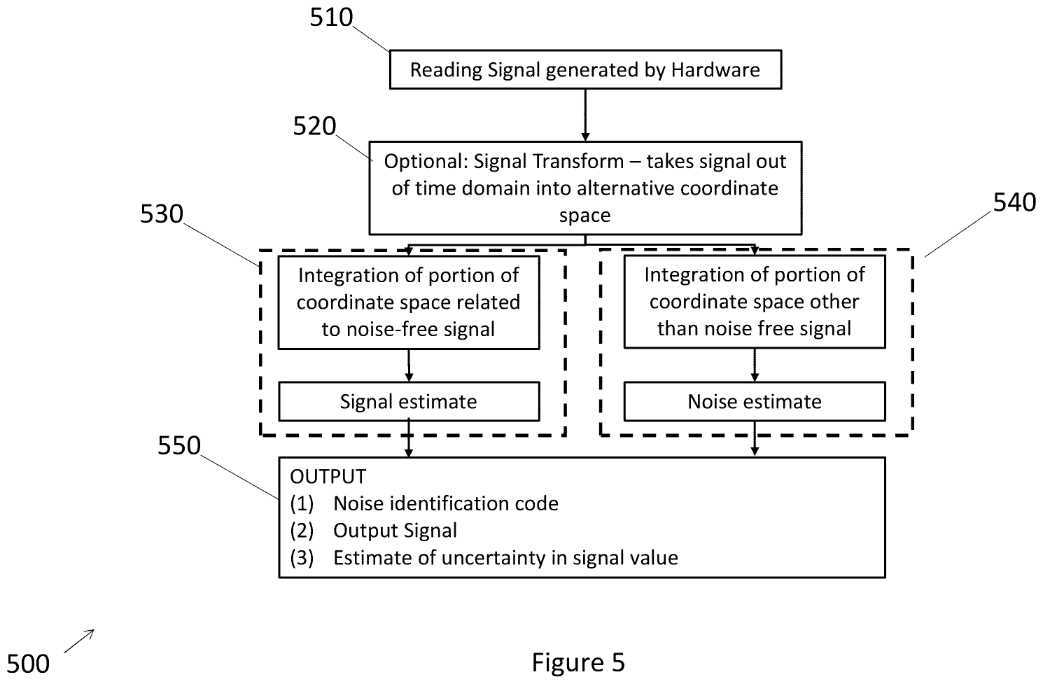

FIG. 5 is a flow chart 500 displaying the main steps that can be implemented in the method of FIG. 1. At step 510, a reading of the detection signal profile is made. The steps 520 to 540 encompass detecting noise, identifying if the detection signal is recoverable and applying a signal recovery algorithm. These are generalized steps and will vary depending on the exact signal recovery algorithm that is being used. Example signal recovery algorithms that can be used will be described in more detail below. At step 550 an output is provided. The output is comprised of three elements. The first is the noise identification code which provides the location in the signal profile that the noise was detected. The second is the reading of the recovered detection signal and the third is the estimated uncertainty in that reading.

Steps 520 to 540 of FIG. 5 will now be described in more detail with reference to example signal recovery algorithms that can be used.

The first example signal recovery algorithm that could be used is a low-pass filtering algorithm. In this algorithm, a low pass filter is applied to obtain the low frequency component and the high frequency component of the signal. The low passed signal will have the following profile:

$${y}_{low , n} = {\sum }_{m = - M}^{M} {y}_{n + m} {filter}_{m} \qquad ( 2 )$$

And the high passed signal will have the following profile:

$${y}_{high , n} = {y}_{n} - {y}_{low , n} \qquad ( 3 )$$

Where the low pass filter has 2M+1 elements and yₙ is the detection signal that was read at step 510. In the simplest case, the noise contamination is represented by the high frequency component, y_{high,n}. The profile of equation (2) is a slightly distorted version of the underlying detection signal profile, for example the electrochemical sensor signal. Use of the low pass filter causes the distortion. Even if there is no noise in the signal there will still be a very slight difference between the input and the filtered output. The degree of distortion to the signal can be assessed using the high frequency component. The low frequency signal y_{low,n} will be passed to the output 550 with the noise contaminated parts of the signal profile replaced through an interpolation function. The interpolation function is derived from portions of the detection signal read at 510 that are not contaminated with noise. This output signal is integrated to give the reading as shown in equation (1).

Another example of a signal recovery algorithm that can be used is template fitting. This algorithm includes the steps of splitting the signal into several windows and restoring the signal in one or more windows based on a template. The template, or templates, can be taken from a look-up table covering a parameterized set of factors. These factors could include, for example, the age of the sensor being used. The template or templates can be derived either from an uncontaminated detection signal profile from the sensor being used or derived from the detection signal profiles of a set of reference sensors. The detection signal that is read in step 510 will be split into a total pre-defined number N of windows, where each window is a small portion of the signal. The template is also split into the same N windows and for each window a correlation calculation is performed by comparing the read detection signal profile with the template signal profile to give a correlation number Rₙ for each window. The correlation calculation used could be, for example, a piecewise correlation function. If the detection signal read at step 510 is noise free, then Rₙ will be the same across the N windows. However, for windows with noise then Rₙ will deviate significantly. A value of Rₙ=1 indicates there is a perfect correlation. A value of Rₙ<0.95 means that replacing the segment potentially introduces up to a 5% deviation from the true signal after fitting. For example, if window n contains noise, then Rₙ for window n will deviate greatly from the Rₙ values in the remaining N−1 windows. The signal in window n will then be restored. Restoration of the signal could be performed by replacing the signal in window n with the related window in the template. The uncertainty of the reading as provided in the output step 550 increases concomitantly with the number of windows M that need to have their signal restored. As an alternative to splitting the detection signal and the template into N pre-defined windows, one can move a time-window over the detection signal and template and calculate the correlation function R (t) to identify time-windows where the detection signal needs to be restored. Scanning a window has the potential to replace a noisy segment which may otherwise occupy two segments in a static N-windowed detection model.

A third example of a signal recovery algorithm is to use a temporal transform as indicated at step 520 of FIG. 5. The temporal transform could be, for example, a Fast Fourier Transform (FFT) or a Wavelet Transform. In the example of utilizing an FFT, a detection signal not contaminated with noise will predominantly occupy low frequency components with some higher frequency components that scale directly with the corresponding low frequency ones. This provides a frequency signature for the detection signal. In a contaminated detection signal, the noise will occupy the higher frequency bands which allows signal repair in frequency space. In frequency space the reading of the signal profile, as provided at step 550, is calculated using the following:

$$Reading = Δ f {\sum }_{n = 1}^{N} {Y}_{n} \qquad ( 4 )$$

Where Yₙ is the signal in the nth frequency bin and N is the total number of frequency bins. If Δt is the overall sample length, then Δf is the smallest frequency interval of the output FFT where it is assumed that the transform takes Z samples and Z is a power of 2. In a simple application of FFT as used in a signal recovery algorithm, the value of N will be set such as to exclude the highest frequency bands and hence exclude the bands likely to be contaminated with noise. The FFT technique can be modified to the Short Term Fast Fourier Transform (STFFT). In the case of STFFT, at step 520 the signal is split into a number of intervals over each of which the FFT is performed. The resultant frequency space signal evolves with time across the intervals.

As in the case of the FFT the STFFT output relevant to the signal will be present in the lower frequency components. Also, as in the FFT case the lowest resolvable frequency is dictated by the size of the interval across which the STFFT is applied. Thus, if the noise has frequency of 5 Hz, the interval length required for both the FFT and STFFT to reject that noise would have to be 0.4 seconds or more as this would make 5 Hz the fourth frequency-bin entry in the returned FFT. Ideally the targeted noise will be present in as high a frequency-bin as possible in the STFFT output to enable rejection whilst maintaining the required time interval to monitor frequency changes over time.

Windowing functions, for example a Hanning window, may be used to avoid edge effects generating high frequency noise. If STFFT is used, then most of the information carried in the detection signal is contained in the zeroth and first order transformation for each time interval. The choice of time-interval used, and interval-overlap is dictated by a combination of several factors including: the frequency range of the noise interference, the effectiveness of noise rejection and the computational load generated by analyzing the detection signal. Instead of FFT or STFFT as the temporal transform, a Wavelet Transform could be used at step 520. For Wavelet transformations, the bulk of the uncontaminated signal is contained in the lower frequency wavelet-components. The basis functions for the wavelets are a set of orthogonal functions forming a Hilbert space. An example of orthogonal functions that can be used is Hermite polynomials. For the shape of the signal profile for electrochemical signals, like the one shown in FIG. 3, any set of orthogonal functions which have a function such that the Gaussian of the zeroth order solution is replaced with a skewed function with an off-set zero point will work.

A fourth example of a signal recovery algorithm that could be used involves a transformation of the detection signal into a compressed space of custom functions. The custom functions can be derived by eigen decomposition over a set of known noise free signals and known noisy signals. The size of eigen vectors can be reduced by binning the signal to decrease the effective sampling frequency prior to analysis. A custom function set can describe the detection signal profile starting with the decomposition of the signal over the time interval of interest t_{crit} into a minimum number eigen vectors and repeat the decomposition over two shorter time segments of t_{crit}/2, then four time segments of t_{crit}/4 and so on until we have 2N segments of t_{crit}/2N. At each of the N steps in the decomposition the remainder of the undescribed signal in each segment can be measured against some criterion and a decision can be taken on whether the next decomposition step for that segment is to be performed. If the zeroth order of the custom function accurately fits the signal profile, then the detection signal is not contaminated by noise. However, in practice it is expected there will always be some very low level background noise. Therefore, a threshold value can be chosen which sets the minimum level of acceptable deviation between the custom function and the detection signal. Any deviation above this threshold value will then indicate that the detection signal is contaminated with noise interference. The noise may arise from, for example, the communications interface. For noise contamination, as shown in FIGS. 4A, 4B and 4C, the full fit of the custom function to the detection signal will only occur when the last segment for the fit is significantly smaller than the entire duration of the detection signal.

The advantage seen in using either the piecewise correlation for template fitting or the custom wavelet fit over a low pass filter is that there is less filter distortion of the underlying electro-chemical detection signal as the template or the zeroth order function match the shape of the electrochemical response. However, these techniques are computationally more intensive. Consequently, it may be decided to perform only the first few orders of the wavelet fit per time interval for the wavelet solution or to use a smoothness measure prior to applying the piecewise correlation to appropriate time intervals. This second option would require high pass filtering of the signal and exclusion of time-intervals with high-passed signal above a threshold. The use of a noise free signal as a template or as the zeroth order function in a Hermite-like polynomial set makes the approach somewhat equivalent to template fitting. However, the approaches diverge when the signal is decomposed further through increasing the order of the polynomial and reducing time interval over which the decomposition applies as a complete description of the signal is obtained.

In all example algorithms given above, the aim is to remove the noise generated by the communications interface, such as the GSM or WiFi transmission, from the electrochemical signal associated with the substance of interest. There exist other denoising filtering approaches and other orthogonal function decomposition approaches.

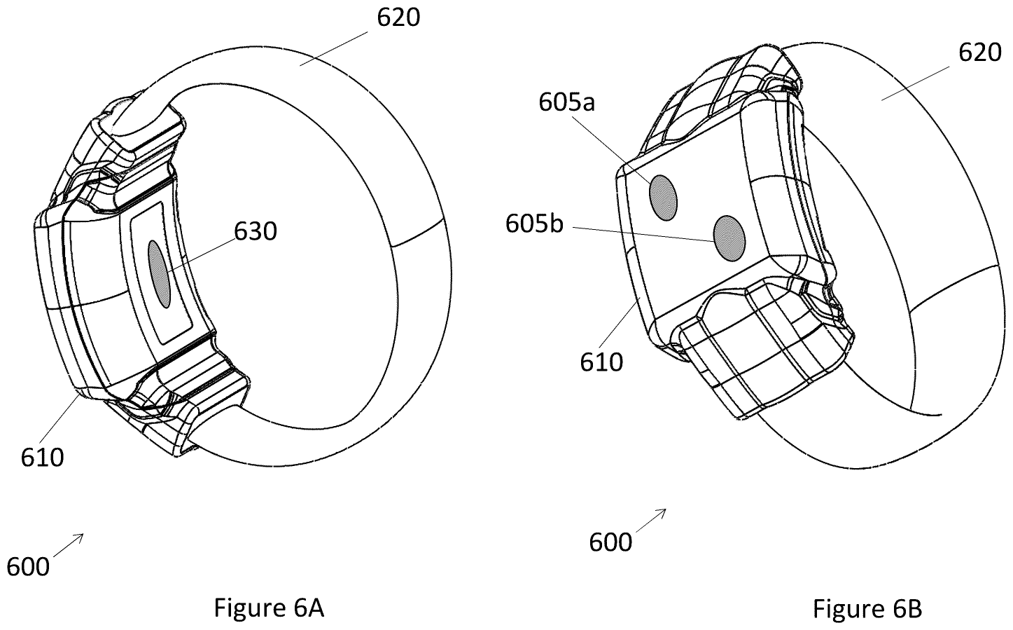

FIG. 6 illustrates a wearable device 600 for implementing the method of FIG. 1. In this example, the wearable device 600 is a tag attachable on a limb of a subject such as the ankle or wrist. FIG. 6A shows the underside of the device to be applied onto a skin portion of the subject, while FIG. 6B shows the front of the device.

The device 600 includes a housing 610 also referred to as an electronic enclosure coupled to a strap 620 for attaching the device to a limb of the subject. The housing 610 has an inner portion to be applied to a skin region of the subject and an outer portion facing outwardly. The inner portion is provided with a diffusion membrane 630 for sampling air in proximity to the skin of the subject. The diffusion membrane 630 is preferably a waterproof membrane, for instance an expanded PTFE membrane. The outer portion is provided with a first port 605a, also referred to as an intake port, and a second port 605b, also referred to as an outlet port.

A sensor, not shown, is provided within the housing 610 and extending between the first port 605a and the second port 605b. The sensor is configured to sense a detection signal for detecting a substance ingested by the subject, wherein the detection signal has a signal profile formed of a peak and a decaying slope. The wearable device 600 may be provided with a communication interface transmitting the detection signal to a remote server. The communication interface may be implemented in various fashions. For instance, the communication interface may be adapted to send and/or receive data via a communication network such as a phone network or a computer network. The phone network may be a mobile network or a landline network. The communication interface may include a transmitter such as an RF transmitter to relay data over a telephone line or directly to the mobile phone network. The communication interface may also be configured to communicate with wireless local area network (LAN) such as a Wi-Fi LAN or through GSM. The communication interface may also include a receiver for receiving data from a remote server.

In another embodiment, the wearable device 600 may include a processor coupled to the sensor. The processor may be configured to read the detection signal, detect noise in the detection signal, identify if the detection signal is recoverable based on the location of the noise along the signal profile and apply a signal recovery algorithm to recover the detection signal. The housing may also contain a geolocation tracker for detecting the position of the wearable device 600. For instance, the housing may be provided with a GNSS or a GPS module, or a combination of both. The strap 620 may be provided with a tamper detection system adapted to detect the removal or interference with the wearable device 600.

It will be appreciated that in other embodiments the wearable device may be designed for use on a specific region of the subject body, which may or may not be a limb. In this case the housing and attachment mechanism may be adapted to fit a particular shape of the chosen body region.

A skilled person will therefore appreciate that variations of the disclosed arrangements are possible without departing from the disclosure. Accordingly, the above description of the specific embodiments is made by way of example only and not for the purposes of limitation. It will be clear to the skilled person that minor modifications may be made without significant changes to the operation described.

1. A method of processing a detection signal contaminated with noise, wherein the detection signal is a signal for detecting a substance ingested by a subject; the method comprising:
reading the detection signal, wherein the signal has a signal profile formed of a peak and a decaying slope;
detecting noise in the detection signal;
identifying if the detection signal is recoverable based on one or more characteristics of the noise in the signal profile; and
applying a signal recovery algorithm to recover the detection signal.

2. The method as claimed in claim 1, wherein the signal recovery algorithm comprises applying a low pass filter to obtain a low frequency component and a high frequency component.

3. The method as claimed in claim 1, wherein the signal recovery algorithm comprises
splitting the detection signal into several windows;
comparing the signal profile of the detection signal with a template signal in each window;
calculating in each window a correlation number; and
restoring the signal in any window in which the correlation number is less than a threshold value.

4. The method as claimed in claim 3, wherein a series of correlation numbers are calculated using a piecewise correlation function.

5. The method as claimed in claim 3, wherein the detection signal is restored in a window by replacing the detection signal in that window with the template signal in that window.

6. The method as claimed in claim 1, wherein the signal recovery algorithm comprises:
performing a temporal transform of the detection signal from a time domain to a frequency domain to obtain a frequency converted signal comprising a set of frequency bands ordered from a lowest frequency to a highest frequency; and
removing a set of preidentified frequency bands from the frequency converted signal.

7. The method as claimed in claim 6, wherein the temporal transform is a Fast Fourier Transform or a Wavelet Transform.

8. The method as claimed in claim 1, wherein the signal recovery algorithm comprises fitting a custom function to the detection signal to obtain a level of noise contamination in the detection signal.

9. The method as claimed in claim 1, comprising assigning a label to the noise or to the detection signal, wherein the label indicates at least one of a location of the noise along the signal profile, information indicating whether the detection signal is recoverable or not recoverable, and a level of accuracy of a recovered signal.

10. The method as claimed in claim 1, wherein noise characteristics comprise noise location and noise intensity.

11. The method as claimed in claim 10, wherein the detection signal is recoverable if the noise is located outside of a predefined window.

12. The method as claimed in claim 1, wherein the detection signal is an electrochemical signal.

13. The method as claimed in claim 1, wherein the substance is alcohol.

14. The method as claimed in claim 1, wherein the noise comprises interference arising from communication devices.

15. The method as claimed in claim 1, comprising estimating an uncertainty of the recovered signal.

16. A monitoring system for use on a subject, the monitoring system comprising:
a monitoring device for use on a subject comprising a sensor configured to sense a detection signal for detecting a substance ingested by the subject, wherein the detection signal has a signal profile formed of a peak and a decaying slope; and a communication interface; and
a processor configured to process the detection signal according to the method of claim 1.

17. The monitoring system as claimed in claim 16, wherein the processor is provided as part of the monitoring device.

18. The monitoring system as claimed in claim 17, wherein the communication interface is configured to transmit the recovered detection signal to a remote server.

19. The monitoring system as claimed in claim 16, wherein the processor is provided on a remote server, the communication interface being configured to transmit raw data to the remote server for processing.

20. The monitoring system as claimed in claim 16, comprising a geolocation tracker configured to provide a location signal of the monitoring device, and/or wherein the communications interface comprises a wireless communications interface.
